(19) United States
(12) Patent Application Publication
(10) Pub. No.: US 2014/0288108 A1
(43) Pub. Date: Sep. 25, 2014

(54) PROCESS FOR THE PREPARATION OF SOLID DISPERSION OF LOPINAVIR AND RITONAVIR

(71) Applicant: RANBAXY LABORATORIES LIMITED, New Delhi, Delhi (IN)

(72) Inventors: Ravindra Tiwari, Kota (IN); Ravindra Agarwa, Udaipur (IN); Janaki Raman Kailya, Cuddalore (IN); Ravi Kochhar, Gurgaon (IN)

(73) Assignee: RANBAXY LABORATORIES LIMITED, New Delhi, Delhi (IN)

(21) Appl. No.: 14/360,465

(22) PCT Filed: Nov. 28, 2012
(86) PCT No.: PCT/IB2012/056809

§ 371 Date: May 23, 2014

(30) Foreign Application Priority Data
Nov. 28, 2011 (IN) 3410/DEL/2011

(51) Int. Cl. A61J 3/00; A61K 31/427; A61K 31/513

(52) U.S. Cl. CPC A61J 3/00; A61K 31/513; A61K 31/427
USPC 514/274

(57) ABSTRACT

An extrusion process for preparation of solid dispersion of lopinavir and ritonavir carried out in twin screw extruder.

### FIELD OF THE INVENTION

The present invention relates to a process for the preparation of a solid dispersion of lopinavir and ritonavir.

### BACKGROUND OF THE INVENTION

The HIV protease inhibitors are a class of antiretroviral agents that competitively inhibit the HIV proteinase or protease enzyme. These are peptide-like molecules that mimic the gag-pol protein, binding onto HIV proteases to prevent the accumulation of structural proteins required for a new virion formation. The HIV protease inhibitors have contributed greatly to the reductions in HIV-associated morbidity and mortality over the last decade and remain a cornerstone of Highly Active Antiretroviral Therapy (HAART). Ritonavir is one of the prominent members of this class of compounds, which is commercialized as Norvir® oral solution and soft gelatin capsules by Abbott Laboratories in the USA. U.S. Pat. Nos. 5,541,206 and 5,648,497 disclose ritonavir and describe its use as an inhibitor of the HIV protease enzyme. Lopinavir is a protease inhibitor described specifically in U.S. Pat. No. 5,914,332. Ritonavir is also available as a co-formulated composition with lopinavir, under the proprietary names Kaletra® and Aluvia® as soft gel capsules and tablets from Abbott Laboratories, USA. U.S. Pat. Nos. 6,037,157 and 6,703,403 disclose the use of ritonavir in combination with an HIV protease inhibitor. Ritonavir is dosed as a pharmacokinetic enhancer with amprenavir, atazanavir, fosamprenavir, lopinavir, saquinavir, tipranavir, darunavir, and the like.

Tablets comprising lopinavir and ritonavir in the form of a solid dispersion proved to be the most stable dosage form developed in the art. U.S. Publication No. 2005/0084529 (the '529 application) discloses a solid pharmaceutical dosage form which comprises a solid dispersion of lopinavir and ritonavir. Solid dispersion provides suitable oral bioavailability and stability to the dosage form. The solid dispersions disclosed in the '529 application are prepared by the process of extrusion using an extruder.

Lopinavir-ritonavir combination formulations always have a certain amount of related substances as an impurity. These related substances are generated due to the process followed for the preparation of the formulations. Certain instrumental and process parameters are responsible for an increased amount of related substances in the final drug formulation. The processes for the preparation of lopinavir-ritonavir combination formulations disclosed in the art involve simple extrusion methods which do not provide any means to control the amount of related substances in the final formulation. Hence, there remains a need to develop a process for the preparation of lopinavir-ritonavir formulations which have a very low level of related substances.

### SUMMARY OF INVENTION

In one general aspect, there is provided an extrusion process for the preparation of a solid dispersion of lopinavir and ritonavir carried out in a twin screw extruder comprising a feeding and a conveying section having the length of about 40% to about 80% of the entire length of the shaft, a mixing section having the length of about 4% to about 20% of the entire length of the shaft, and a discharge section having the length of about 10% to about 30% of the entire length of the shaft, wherein the process is carried out at screw speed from about 100 RPM to about 650 RPM.

In another general aspect, there is provided an extrusion process for the preparation of a solid dispersion of lopinavir and ritonavir carried out in a twin screw extruder comprising a feeding and a conveying section having the length of about 40% to about 80% of the entire length of the shaft, a mixing section having the length of about 4% to about 20% of the entire length of the shaft, and a discharge section having the length of about 10% to about 30% of the entire length of the shaft, wherein the process is carried out at screw speed from about 100 RPM to about 650 RPM, and wherein the mixing section has mixing zones angles between 0° to 120°.

In another general aspect, there is provided an extrusion process for the preparation of a solid dispersion of lopinavir and ritonavir carried out in a twin screw extruder comprising a feeding and a conveying section having the length of about 40% to about 80% of the entire length of the shaft, a mixing section having the length of about 4% to about 20% of the entire length of the shaft, and a discharge section having the length of about 10% to about 30% of the entire length of the shaft, wherein the process is carried out at screw speed from about 100 RPM to about 650 RPM, and wherein the mixing section has mixing zones angles between 0° to 120° and wherein the process is carried out at a feed rate of from about 5 Kg/hr to about 30 Kg/hr, and the processing temperature from about 100° C. to about 140° C.

In another general aspect, there is provided an extrusion process for the preparation of a solid dispersion of lopinavir and ritonavir carried out in a twin screw extruder comprising a feeding and a conveying section having the length of about 40% to about 80% of the entire length of the shaft, a mixing section having the length of about 4% to about 20% of the entire length of the shaft, and a discharge section having the length of about 10% to about 30% of the entire length of the shaft, wherein the process is carried out at screw speed from about 100 RPM to about 650 RPM, and wherein the mixing section has mixing zones angles between 0° to 120° and wherein the process is carried out at a feed rate of from about 5 Kg/hr to about 30 Kg/hr, and the processing temperature from about 100° C. to about 140° C., and wherein the solid dispersion has less than 0.2% of N-methyl-1-[2-(propan-2-yl)-1,3-thiazol-4yl]methanamine, as impurity having molecular weight 170 (MW 170 ), less than 0.2% of Impurity B and less than 0.5% of Impurity F.

### DETAILED DESCRIPTION OF THE INVENTION

The process of the present invention helps in controlling the amount of related substances at the lowest level in the final formulations. The solid dispersion was prepared under controlled instrumental and process parameters, which significantly limited the amount of related substances in the formulation. The process does not involve any extra cost and the formulations were found to have a significantly low amount of related substances.

The term “lopinavir”, as used herein, includes a free lopinavir base as well as its pharmaceutically acceptable salts. Lopinavir is chemically designated as (2S)-N-[(2S,4S,5S)-5-[[2-(2,6-dimethylphenoxy)acetyl]amino]-4-hydroxy-1,6-diphenylhexan-2-yl]-3-methyl-2-(2-oxo-1,3-diazinan-1-yl)butanamide.

The term “ritonavir”, as used herein, includes a free ritonavir base as well as its pharmaceutically acceptable salts. Ritonavir inhibits the CYPSA-mediated metabolism of lopinavir, thereby providing increased plasma levels of lopinavir. Ritonavir is chemically designated as 1,3-thiazol-5-ylmethyl N-[(2S,3S,5S)-3-hydroxy-5-[[(2S)-3-methyl-2-[[methyl-[(2-propan-2-yl-1,3-thiazol-4-yl)methyl]carbamoyl]amino]butanoyl]amino]-1,6-diphenylhexan-2-yl]carbamate.

The term “solid dispersion”, as used herein, refers to a group of solid formulations generally consisting of a pharmaceutically acceptable carrier matrix, lopinvair, and ritonavir homogeneously dispersed therein. The matrix may be either crystalline or amorphous. The drug may be dispersed molecularly, in amorphous particles (clusters), or in crystalline particles.

The “pharmaceutically acceptable carrier”, as used herein, refers to both polymeric and non-polymeric carriers; hydrophilic and hydrophobic carriers that are capable of dissolving and/or dispersing one or more of the HIV-protease inhibitor(s) and includes homopolymers and copolymers of N-vinyl lactams, e.g., N-vinyl-2-pyrrolidone, crosslinked N-vinyl-2-pyrrolidone, copolymer of N-vinyl-2-pyrrolidone and vinyl acetate (copovidone); cellulose esters and cellulose ethers, e.g., hydroxypropyl methylcellulose, hydroxypropyl cellulose, hydroxyethyl cellulose; cellulose phthalates or succinates; polyacrylates and polymethacrylates; polyacrylamides; vinyl acetate polymers; high molecular weight polyalkylene oxides such as polyethylene oxide; polyethylene glycols; cyclodextrin, oligo- and polysaccharides such as xanthan gum; and combinations thereof. Copolymer of N-vinyl-2-pyrrolidone and vinyl acetate (copovidone) such as those which are available as Plasdone® or Kollidon® from ISP and BASF, respectively, may be used as the pharmaceutically acceptable carrier. The amount of the pharmaceutically acceptable carrier may vary from about 1% to about 99% by weight of the unit dosage form, more particularly from about 50% to about 85% by weight of the unit dosage form.

The solid dispersion composition may also include one or more of pharmaceutically acceptable excipients, for example, fillers, disintegrants, glidants, lubricants, surfactants, and combinations thereof.

Fillers may be selected from saccharides such as lactose, dextrose, sucrose, fructose, maltose; sugars such as mannitol, erythritol, sorbitol, xylitol and lactitol; cellulose derivatives such as powdered cellulose, and microcrystalline cellulose; dicalcium phosphate; tribasic calcium phosphate; calcium sulphate; calcium carbonate; kaolin; starch and starch derivatives such as pregelatinized starch, partially pregelatinized starch; and cellulose ethers such as carboxymethyl cellulose, methylcellulose, hydroxypropyl cellulose, and hydroxypropyl methylcellulose; carboxy vinyl polymers such as carbomers; acrylates such as Eudragit®'s; polyvinylpyrrolidone; xanthan gum; guar gum; and other such materials routinely used in the art of solid dosage form manufacturing.

Disintegrants may be selected from croscarmellose sodium, sodium starch glycolate, crosslinked polyvinylpyrrolidone, corn starch, potato starch, pregelatinized starch, low-substituted hydroxypropylcellulose, alginates, carboxymethyl starches, methacrylic acid divinylbenzene copolymer salts, and microcrystalline cellulose.

Lubricants and/or glidants that may be used include magnesium stearate, calcium stearate, zinc stearate, sodium stearyl fumarate, powdered stearic acid, magnesium oleate, calcium palmitate, potassium laureate, sodium suberate, vegetable oil, mineral oil, talc, colloidal silicon dioxide, and corn starch.

Pharmaceutically acceptable surfactants may include polyoxyethylene alkyl ethers, e.g., polyoxyethylene lauryl ether, polyoxyethylene cetyl ether, polyoxyethylene stearyl ethers; polyoxyethylene alkylaryl ethers, e.g., polyoxyethylene nonylphenyl ethers, polyoxyethylene octylphenyl ethers; polyethylene glycol fatty acid esters, e.g., PEG-200 monolaurate, PEG-200 dilaurate, PEG-300 dilaurate, PEG-400 dilaurate, PEG-300 distearate, PEG-300 dioleate; alkylene glycol fatty acid mono esters, e.g., propylene glycol monolaurate; sucrose fatty acid esters, e.g., sucrose monostearate, sucrose distearate, sucrose monolaurate, sucrose dilaurate; sorbitan fatty acid mono esters such as sorbitan monolaurate (Span 20), sorbitan monooleate, sorbitan monopalmitate (Span 40), or sorbitan stearate; polyoxyethylene castor oil derivates, e. g., polyoxyethyleneglycerol triricinoleate or polyoxyl-35 castor oil (Cremophor® EL; BASF Corp.); polyoxyethyleneglycerol oxystearate such as polyethylenglycol 40 hydrogenated castor oil (Cremophor® RH 40) or polyethylenglycol 60 hydrogenated castor oil (Cremophor® RH 60); or block copolymers of ethylene oxide and propylene oxide, also known as polyoxyethylene polyoxypropylene block copolymers or polyoxyethylene polypropyleneglycol, such as Poloxamer® 124, Poloxamer® 188, Poloxamer® 237, Poloxamer® 388, Poloxamer® 407 (BASF Wyandotte Corp.); polyglycolized glycerides, for example, lauroyl macrogolglycerides (Gelucire® 44/14), stearoyl macrogolglycerides (Gelucire® 50/13); Labrasol® or Transcutol® (Gattefosse Canada Inc.); Vitamin E/TPGS (tocopheryl propylene glycol 1000 succinate, sold by Eastman); polyethylene glycol 15 hydroxystearate (Solutol® HS 15 sold by BASF); or a mono fatty acid ester of polyoxyethylene (20) sorbitan, e. g., polyoxyethylene (20) sorbitan monooleate (Tween® 80), polyoxyethylene (20) sorbitan monostearate)(Tween®), polyoxyethylene (20) sorbitan monopalmitate (Tween® 40), polyoxyethylene (20) sorbitan monolaurate (Tween® 20); or mixtures of one or more thereof. In one embodiment, the pharmaceutically acceptable surfactant is sorbitan monolaurate. The amount of a pharmaceutically acceptable surfactant may vary from about 1% to about 10% by weight of the solid dispersion.

One or more of the solvents may be used to dissolve and/or disperse the pharmaceutically acceptable carrier, lopinavir, ritonavir, and/or the pharmaceutically acceptable surfactant. Such solvents or the solutions/dispersions formed may further be used as granulating solvents. Such solvents include one or more of alcohols, e.g., isopropyl alcohol; aliphatic hydrocarbons, e.g., acetone; and esters.

Optionally, the solid dispersion obtained by the process may be milled or ground to granules. The granules may then be compacted. Compacting is a process whereby a powder mass comprising the granules is densified under high pressure in order to obtain a compact with low porosity, e.g., a tablet.

The term “melt extrusion”, as used herein, comprises the steps of preparing a homogeneous melt of the lopinavir and ritonavir, the pharmaceutically acceptable carrier, and the surfactant, and cooling the melt until it solidifies. “Melting” is a transition of a solid into a liquid or rubbery state in which it is possible for one component to get embedded homogeneously in the other. Melting usually involves heating above the softening point of the pharmaceutically acceptable carrier. Usually, the melt temperature is in the range of about 70° C. to about 250° C., preferably from about 80° C. to about 180° C., most preferred from about 100° C. to about 140° C.

The melt-extrusion process may be carried out in an extruder. Suitable extruders include single screw extruders, intermeshing screw extruders, or multi screw extruders, preferably twin screw extruders. The “twin screw extruder” comprises at least two rotating shafts. Each of the shafts carries feeding and conveying, mixing and discharging sections axially one behind the other and the shafts are corotating. Each section is defined by screw elements which are arranged in specific screw configurations. Screw configuration is the combination of a specific number of screw elements of specific length which are combined in a specific sequence and a specific mixing zone angle. Each section has a specific length. The number of screw elements present in that particular section defines the total length of that section. The total length of the screw shaft is about 960 mm. The length and diameter of the different screws present in the different sections of the extruder may be in the range shown below:

| Different Screws | Length (mm) | L-D correlation |
|---|---|---|
| Extrusion Screw | 36 | (L = 1.5D) |
| Transition Screw | 6 | (L = D/4) |
| Feed Screw | 24 | (L = D) |
| Screw Element | 6 | (L = D/4) |
| Long Pitched Screw | 48 | (L = 2D) |
| Short Pitched Screw | 24 | (L = D) |

The feeding and conveying section is positioned farthest upstream, close to the hopper of the extruder, while the mixing section is positioned downstream of the feeding and conveying section. There are two mixing zones made up of mixing elements. A part of the conveying section composed of conveying elements is present between the two mixing zones, and the discharging section is positioned farthest downstream, close to the discharge opening of the extruder. The feeding and conveying element as well as the discharging element allows a smooth passage of the material fed to the extruder from the feed end to the discharge end of the extruder.

The mixing section comprises the mixing element “being derived from a screw element” and is intended to mean an element whose basic shape is that of a screw element, but which has been modified such that it exerts a compounding or mixing effect in addition to the conveying effect. The screw type element may be right handed or left handed, or a combination thereof. The mixing exerted by the mixing may be distributive or dispersive.

The mixing zone angles are the angles between a screw element in the mixing zone considering the last screw element in the second mixing zone as 0° in a clock-wise direction as the view from man machine side. Mixing zone angles may be between 0° to 120°.

“Screw speed”, as used herein, is the rotation rate of the screw when the instrument is functional. The screw speed is measured in RPM units. The screw speed may be in the range of about 100 RPM to about 650 RPM.

“Feed rate” is the rate at which the starting material to be extruded enters into the barrel from the hopper attached in the feeding section. The feed rate is measured in Kg/hr. The feed rate may be in the range of about 5 Kg/hr to about 30 Kg/hr.

“Processing temperature”, as used herein, is the temperature of Zone 6, Zone 7, Zone 8, and the die. The extruder barrel comprises several heating zones which maintain temperature throughout the different sections of the extruder barrel. The extruder comprises a total of 9 zones. Each zone is maintained with a desired temperature. The temperature distribution of the different zones of the extruder may be in the range shown below:

| | Zones | | | | | | | | |
|---|---|---|---|---|---|---|---|---|---|
| | Zone 1 | | | | Zone 5 | | | | |
| | (Back | | | | (First | Zone 6 | Zone 7 | Zone 8 | Die |
| | Chill | | | | Mixing | Second Mixing | | Discharge | |
| | Unit) | Zone 2 | Zone 3 | Zone 4 | Zone) | Zone | | section | |
| Temperature | 15-25 | 25-35 | 35-65 | 65-95 | 95-105 | 105-125 | 110-130 | 110-130 | 110-130 |
| (° C.) | | | | | | | | | |

Controlled temperature distribution helps to get homogeneous, smooth, and transparent extrudate which, in particular, has not been damaged by temperatures too high for the active ingredient.

The active ingredients can be employed as such or as a solution or dispersion in a suitable solvent such as alcohols, aliphatic hydrocarbons, or esters. The solvent is removed, e.g., evaporated, upon preparation of the melt.

Impurity B and Impurity F are the related substances or impurities which may be present in the lopinavir-ritonavir solid dispersion. Impurity B is thiazol-5-ylmethyl[(1S,2S,4S)-4-[[(2S)-2amino-3-methylbutanoyl]amino]-1-benzyl-2-hydroxy-5-phenylpentyl]carbamate and Impurity F is thiazol-5-ylmethyl [(1S,2S,4S)-1-benzyl-4-[(2S)-1-benzyl-2-hydroxy-4-[(4S)-4-(1-methylethyl)-2,5-dioxoimidazolidin-1-yl]-5-phenylpentyl]carbamate.

In one of the embodiments, there is provided an extrusion process for the preparation of a solid dispersion of lopinavir and ritonavir carried out in a twin screw extruder, comprised of a feeding and a conveying section having the length of about 55% to 75% of the entire length of the shaft, a mixing section having the length of about 5% to 15% of the entire length of the shaft, and a discharge section having the length of about 15% to 25% of the entire length of the shaft, wherein the process is carried out at a screw speed of from about 100 RPM to about 650 RPM; more preferably from about 150 RPM to about 400 RPM.

In another embodiment, there is provided an extrusion process for the preparation of a solid dispersion of lopinavir and ritonavir carried out in a twin screw extruder, wherein the feeding and the conveying section comprises 16 feed screws (24 mm×16=384 mm size), 2 long pitched feed screws (48 mm×2=96 mm size), and 1 short pitched screw (24 mm×1=24 mm size); the mixing section comprises 8 screw elements in the first mixing zone (6 mm×8=48 mm size) and 7 screw elements in the second mixing zone (6 mm×7=42 mm size); 6 feed screw (24 mm×6=144 mm) forming a part of the conveying section are present between the first and the second mixing zones and the discharge section comprises 1 extrusion screw (36 mm×1=36 mm size), 1 transition element (6 mm×1=6 mm size), 7 feed screw (24 mm×7=168 mm size), and 1 feed screw (12 mm×1=12 mm size).

In another embodiment, there is provided an extrusion process for the preparation of a solid dispersion of lopinavir and ritonavir carried out in a twin screw extruder wherein the mixing section has 8 screw elements from 1^{st} to 8^{th} downstream in the first mixing zone having angles of 120°, 30°, 120°, 30°, 120°, 30°, 90°, and 120°, respectively; and 7 screw elements 1^{st} to 7^{th} downstream in the second mixing zone having angles of 120°, 0°, 60°, 120°, 0°, 90°, and 0° respectively.

In another embodiment, there is provided an extrusion process for the preparation of a solid dispersion of lopinavir and ritonavir carried out in a twin screw extruder wherein the process is carried out at a feed rate of from about 5 Kg/hr to about 30 Kg/hr; more preferably, 10 kg/hr to 20 Kg/hr, and the process temperature from about 100° C. to about 140° C.; more preferably about 110° C. to about 120° C.

In another embodiment, there is provided an extrusion process for the preparation of a solid dispersion of lopinavir and ritonavir carried out in a twin screw extruder wherein the solid dispersion of lopinavir and ritonavir comprises less than 0.165% of MW 170, less than 0.044% of Impurity B and less than 0.251% of Impurity F.

From the above, it is apparent that various modifications and combinations of the processes detailed in the text may be made without departing from the spirit and scope of the invention. The invention, as described herein, may be illustrated by the following examples but they should not be construed as limiting the scope of the invention in any way.

The term “about” as used herein mean a variation off ±10%.

### EXAMPLES

Lopinavir-ritonavir solid dispersion has a composition as shown in Table I.

TABLE I
Lopinavir-Ritonavir solid dispersion (tablet composition)

| Ingredients | Quantity (mg/tablet) |
|---|---|
| Intragranular (solid dispersion) | |
| Lopinavir | 200.00 |
| Ritonavir | 50.00 |
| Copovidone | 591.70 |
| Sorbitan laurate | 83.00 |
| Colloidal anhydrous silica | 12.20 |
| Extragranular | |
| Lactose monohydrate | 373.00 |
| Colloidal anhydrous silica | 8.10 |
| Sodium Stearyl Fumerate | 12.00 |
| Core Tablet Weight | 1330.00 |
| Film Coating Material | |
| Opadry ® II Yellow | 33.25 |
| Purified water | q.s. |

The lopinavir-ritonavir solid dispersion of the present invention was prepared by the hot melt extrusion process using a twin screw extruder. Many experimental formulations of the above composition were prepared using different instrumental and process parameters.

### Instrumental Parameters

The qualitative comparison of formulations prepared under different mixing zone angles and mixing zone lengths is represented in Table II.

TABLE II
Qualitative comparison of formulations prepared
under different instrumental parameters

| S. | Instrumental | | | | |
|---|---|---|---|---|---|
| No. | Parameters | Example 1 | Observation | Example 2 | Observation |
| 1 | Overall | 90 mm | Transparent | 78 mm | Translucent |
| | mixing | | Extrudes | | Extrudes |
| | zone length | | | | |
| 2 | Length of | 48 mm | Transparent | 24 mm | Translucent |
| | first | | Extrudes | | Extrudes |
| | mixing zone | | | | |
| 3 | Length of | 42 mm | Transparent | 54 mm | Transparent |
| | second | | Extrudes | | Extrudes |
| | mixing zone | | | | |
| 4 | Mixing zone | 4 elements | Transparent | 2 elements | Translucent |
| | angle at | at 120°, | Extrudes | at 120°, | Extrudes |
| | first | 3 elements | | 2 elements | |
| | mixing zone | at 30°, | | at 30° | |
| | | 1 element | | | |
| | | 90° | | | |
| 5 | Mixing zone | 2 elements | Transparent | 4 elements | Transparent |
| | angle at | at 0°, | Extrudes | at 0°, | Extrudes |
| | second | 2 elements | | 2 elements | |
| | mixing zone | 90°, | | 90°, | |
| | | 2 elements | | 2 elements | |
| | | at 120°, | | at 120°, | |
| | | 1 element | | 1 element | |
| | | at 60° | | at 60° | |

From the formulations prepared under different mixing zone length and different mixing zone angles as shown in Example 1 and Example 2 of Table II, formulations prepared as per Example 1 were finalized for further experimentation. Finalized instrumental parameters of Example 1 for preparation of lopinavir-ritonavir solid dispersion are below:

### Mixing Zone Length

Length of first mixing zone: 48 mm

Length of second mixing zone: 42 mm

Total length of mixing section: 90 mm

### Mixing Zone Angle

First Mixing Zone: 8 screw elements (6 mm size) downstream set as 120°, 30°, 120°, 30°, 120°, 30°, 90°, and 120°

Second Mixing Zone: 7 screw elements (6 mm size) downstream set as 120°, 0°, 60°, 120°, 0°, 90°, and 0°.

### Overall Screw Configuration (Upstream)

1 extrusion screw (36 mm size)—one transition element (6 mm size)—7 feed screws (24 mm size)—1 feed screw (12 mm size)—7 screw elements (6 mm size) upstream set as 0°, 90°, 0°, 120°, 60°, 0°, and 120°—6 feed screws (24 mm size)—8 screw elements (6 mm size) upstream set as 120°, 30°, 120°, 30°, 120°, 30°, 90°, and 120°—16 feed screws (24 mm size)—2 long pitched feed screw (48 mm size)—1 short pitched feed screw (24 mm size).

### Process Parameters

Instrumental parameters as per Example 1 were employed to further prepare lopinavir-ritonavir solid dispersion formulations under different process parameters such as processing temperature, feed rate, and screw speed, as shown in Table III and Table IV.

TABLE III
Process parameters to prepare lopinavir-ritonavir solid dispersion
formulations prepared under instrumental parameters of Example 1.

| | | | Screw speed |
|---|---|---|---|
| Examples | Temperature (° C.) | Feed rate (kg/hr) | (RPM) |
| Example 1.1 | 110 | 10 | 350 |
| Example 1.2 | 130 | 10 | 350 |
| Example 1.3 | 110 | 20 | 350 |
| Example 1.4 | 130 | 20 | 350 |
| Example 1.5 | 110 | 10 | 650 |
| Example 1.6 | 130 | 10 | 650 |
| Example 1.7 | 110 | 20 | 650 |
| Example 1.8 | 130 | 20 | 650 |
| Example 1.9 | 120 | 15 | 500 |
| Example 1.10 | 100 | 15 | 500 |
| Example 1.11 | 140 | 15 | 500 |
| Example 1.12 | 120 | 5 | 500 |
| Example 1.13 | 120 | 25 | 500 |
| Example 1.14 | 120 | 15 | 200 |
| Example 1.15 | 120 | 15 | 750 |
| Example 1.16 | 115 | 10 | 250 |
| Example 1.17 | 115 | 10 | 350 |
| Example 1.18 | 115 | 15 | 250 |
| Example 1.19 | 115 | 15 | 350 |
| Example 1.20 | 110 | 20 | 350 |
| Example 1.21 | 115 | 20 | 350 |
| Example 1.22 | 115 | 20 | 300 |
| Example 1.23 | 115 | 10 | 200 |
| Example 1.24 | 115 | 8 | 165 |

TABLE IV
Related Substance data of formulations prepared under
process parameters of Example 1.1 to Example 1.24.

| | | % Weight of Related Substances | | |
|---|---|---|---|---|
| Examples | | Impurity MW 170 | Impurity B | Impurity F |
| Example 1.1 | | 0.196 | 0.088 | 0.367 |
| Example 1.2 | | 0.247 | 0.090 | 0.535 |
| Example 1.3 | | 0.166 | 0.097 | 0.333 |
| Example 1.4 | | 0.230 | 0.104 | 0.500 |
| Example 1.5 | | 0.353 | 0.069 | 0.822 |
| Example 1.6 | | 0.443 | 0.070 | 1.030 |
| Example 1.7 | | 0.324 | 0.085 | 0.630 |
| Example 1.8 | | 0.400 | 0.089 | 0.789 |
| Example 1.9 | | 0.291 | 0.084 | 0.544 |
| Example 1.10 | | 0.253 | 0.075 | 0.489 |
| Example 1.11 | | 0.382 | 0.086 | 0.761 |
| Example 1.12 | | 0.343 | 0.071 | 0.961 |
| Example 1.13 | | 0.217 | 0.079 | 0.353 |
| Example 1.14 | | 0.170 | 0.074 | 0.264 |
| Example 1.15 | | 0.406 | 0.068 | 0.900 |
| Example 1.16 | | 0.182 | 0.040 | 0.306 |
| Example 1.17 | | 0.193 | 0.035 | 0.457 |
| Example 1.18 | | 0.164 | 0.044 | 0.251 |
| Example 1.18 | | 0.189 | 0.039 | 0.377 |
| Example 1.20 | | 0.160 | 0.039 | 0.323 |
| Example 1.21 | | 0.191 | 0.041 | 0.342 |
| Example 1.22 | | 0.190 | 0.039 | 0.316 |
| Example 1.23 | | 0.171 | 0.040 | 0.261 |
| Example 1.24 | | 0.127 | 0.039 | 0.249 |
| KALETRA ® | Batch | 0.426 | 0.022 | 1.322 |
| | No. | | | |
| | 6001438 | | | |
| | Batch | 0.504 | 0.023 | 1.390 |
| | No. | | | |
| | 6010107 | | | |

The observed related substance values were found to be within the desired limits. It was also observed that at a high feed rate and a lower screw speed, the extrusion was smooth and the solid dispersion had lesser levels of impurities.

1. An extrusion process for the preparation of a solid dispersion of lopinavir and ritonavir carried out in a twin screw extruder comprising a feeding and a conveying section having length of 40% to 80% of the entire length of a shaft, a mixing section having length of 4% to 20% of the entire length of the shaft, and a discharge section having length of 10% to 30% of the entire length of the shaft, wherein the process is carried out at screw speed from 150 RPM to 650 RPM.

2. The extrusion process according to claim 1, wherein the mixing section has mixing zone angles between 0° to 120°.

3. The extrusion process according to claim 1, wherein the process is carried out at a feed rate of from 5 Kg/hr to 30 Kg/hr, and a processing temperature from 100° C. to 140° C.

4. The solid dispersion according to claim 1, wherein the solid dispersion has less than 0.2% of impurity MW 170, less than 0.2% of Impurity B, and less than 0.5% of Impurity F.

5. (canceled)
